(19) United States
(12) Patent Application Publication
(10) Pub. No.: US 2020/0281848 A1
(43) Pub. Date: Sep. 10, 2020

(54) COMPOSITION CONTAINING MOXIDECTIN FOR TREATING PARASITES INFESTATIONS

(71) Applicant: CEVA SANTE ANIMALE, LIBOURNE (FR)

(72) Inventors: Florence GUIMBERTEAU, LIBOURNE (FR); Hamadi KAREMBE, LIBOURNE (FR)

(21) Appl. No.: 16/765,301

(22) PCT Filed: Nov. 23, 2018
(86) PCT No.: PCT/EP2018/082437

§ 371 Date: May 19, 2020

(30) Foreign Application Priority Data
Nov. 23, 2017 (EP) 17203184.1

(51) Int. Cl. A61K 9/00; A61K 31/366; A61K 47/10; A61K 47/38; A61K 47/14; A61P 33/00

(52) U.S. Cl. CPC A61K 9/0017; A61K 31/366; A61P 33/00; A61K 47/38; A61K 47/14; A61K 47/10

(57) ABSTRACT

The present invention relates to a composition comprising moxidectin or a salt thereof for use in preventing and/or treating a parasite infestation in non-human mammals, in which said composition is topically administered every 3 to 9 months.

### FIELD OF THE INVENTION

The invention relates to a veterinary or pharmaceutical composition comprising moxidectin, or a salt thereof, for use in preventing and/or treating parasites infestations in a non-human mammal, characterized in that the composition is topically administered every 3 to 9 months.

It is also related to a method of treatment and/or prevention of parasites infestations in a non-human mammal, comprising topically administering every 3 to 9 months to said non-human mammal a veterinary or pharmaceutical composition comprising moxidectin, or a salt thereof.

### BACKGROUND OF THE INVENTION

Non-human mammals, for example companion animals such as dogs, puppies, cats, kitten, rabbits, ferrets, horses, and pigs, are often subject to parasites infestations which feed on blood and therefore to infections caused by these parasites. These parasites may be ectoparasites, such as ticks, fleas, scabies, louses/nits, flies, mosquitoes, or endoparasites. Moreover, some of these parasites are intermediary hosts of endoparasites such as flatworms (or plathelminths), hookworms, whipworms or roundworms (or nematodes).

More particularly, heartworm is a parasitic roundworm (especially Dirofilaria immitis) that spreads from host to host through bites of mosquitoes (intermediate host). The definitive and most affected hosts are dogs but it can also infect cats, wolves, coyotes, foxes, ferrets, sea lions and even bovines and humans. It is found in the five continents.

The parasite is commonly called “heartworm” because the adult reproductive stage of its life cycle resides primarily in the right ventricle of its host where it can live for many years. Heartworms infection may result in serious diseases for the host: dirofilariasis, and more precisely, heartworm disease.

When a mosquito bites an infected animal, young heartworms, called microfilariae, enter the mosquito's system. Within two weeks, the microfilariae develop into infective larvae inside the mosquito and these infective larvae can be transmitted to other animals when mosquito bites again.

When entering the dog's blood system via this bite, larvae develop (macrofilariae) and migrate to the dog's heart where they mature and breed. The dirofilaria life cycle is completed when the ingested microfilariae mature into infective larvae in the mosquito. Development of larvae into adult worms takes about 180 days in dogs, while, the life cycle of heartworms is approximately 6 months.

Dirofilaria immitis appears as white threadlike round worms reaching up to 20 cm long for adult males (12-20 cm) and 31 cm for adult females (25-31 cm), with a mean diameter of 1 mm.

Heartworms are primarily found in the pulmonary artery in dogs with low parasitic burden (<50 worms). In infestations with high parasitic burden (>50 worms), worms may reach the right ventricle, right atrium, and occasionally vena cava. The initial response includes swelling of small pulmonary arteries and blood clotting. The physical presence of heartworms in the pulmonary artery and right ventricle of the canine heart, and the resulting destruction of tissue, causes respiratory and circulatory problems which can be fatal under conditions of stress or vigorous exercise. Pulmonary hypertension and right-sided heart failure may result in congestive heart failure.

Because it is necessary to have a lot of heartworms to clog up blood flow to a significant degree, heartworms can be present inside the heart for up to 2 or 3 years before causing clinical signs. As the disease progresses, lung tissue can be destroyed leading to a worsening cough while liver and kidney damages can occur due to reduced blood flow in organs. If left untreated, heartworm disease may result in death. Humans may also become infected as aberrant hosts. But, most infective larvae introduced in human die.

Even though safe, highly effective and convenient prevention strategies have been available for the past two decades, heartworm disease, due to Dirofilaria immitis, continues to cause severe damages and even death in dogs and other mammals (cats, bovines, humans, guinea porcine, and ferrets) in many parts of the world. Moreover, the parasite and vector mosquitoes continue to spread into areas where they have not been reported previously.

Currently only two arsenic derivatives are available for curative treatment for clinically infested dogs. First, thiacetarsamide (Caparsolate®, by Abbott Laboratories) which is an old medication, with severe adverse effects and second, melarsomine dihydrochloride (Immiticide®, by Merial), which is a more recent drug with fewer side effects. For chemoprophylaxis, two alternatives are possible to prevent heartworm disease in dogs: daily administration of diethylcarbamazine citrate, or monthly administration of macrocyclic lactones.

Alike, scabies (Demodex sp., Sarcoptes sp., Otodectes sp., . . . ) are hard to control/kill because only few efficient treatments do exist, and the infected animal has to be frequently treated. For example, ear mites are mites that live in the ears of animals and humans. Ear mites infections in dogs and cats can cause intense itching in one or both ears, and trigger scratching at the affected ear. An unusually dark colored ear wax/cerumen may also be produced. The most common lesion associated with ear mites is an open or crusted skin wound at the back or base of the ear, caused by abrasion of the skin by hind limb claws. This lesion often becomes infected and crusted from ordinary skin bacteria, so that the common presentation of ear mites is such a wound appearing on the back or base of one or both ears. The most common ear mites treatments currently use the antiparasitic selamectin, which is administered as a once-a-month preparation that can be topically applied to the animal's skin, or ivermectin.

The control of such endo- and ectoparasites has long been recognized as an important aspect of human and animal health regimens. Although a number of alternatives to control infestations are in use, they suffer from many problems, including a limited spectrum of activity, the need of repeated treatment (lack of compliance) and, in some rare instances, resistance by parasites, in particular with the use of carbamates, organophosphorous compounds or pyrethroids. That is why it is very important to develop new efficient treatments.

Number of treatments have been commercialized to treat such parasites. For example, chewable tablets containing ivermectin (Heartgard®, by Merial) have been commercialized to prevent canine heartworm disease (6.0 micrograms per kilogram body weight, minimum), by eliminating the tissue stage of heartworm larvae (Dirofilaria immitis) for one month (30 days) after infection. But the treatment has two limitations: it can only be used once-a-month and therefore, has an efficiency of one month only, and it is an oral medication: degradation by liver or gastric juices, delayed effect, and drug interactions.

As another example, a slow release formulation of subcutaneously injected moxidectin-impregnated lipid microspheres, providing continuous protection against dirofilaria of six months following a single dose administration, has been marketed by Zoetis under the name of Moxidectin SR®, ProHeart 6® or Guardian SR®. It has many disadvantages: it is a suspension which requires a complex preparation and use in general veterinary practice and as it is an injectable product, it has to be administered by a veterinarian, with a syringe, which can eventually induce local tolerance issues. Besides, this product has been voluntarily removed from the US market in September 2004 because of safety related issues, and currently has been allowed once again by FDA under a risk minimization and restricted distribution program.

A composition of imidacloprid and moxidectin is commercialized by Bayer (Advocate® or Advantage Multi®) to prevent dirofilariasis, it is topically applied, but imidacloprid can be toxic for some animals (birds for example) and the product has to be administered every month, during several months, no dose must be missed, which is restrictive. Advantage Multi® notice details many drawbacks: “for prevention of heartworm disease, Advantage Multi® for Dogs should be administered at one-month intervals. Advantage Multi® for Dogs may be administered year-round or at a minimum should start one month before the first expected exposure to mosquitoes and should continue at monthly intervals until one month after the last exposure to mosquitoes. If a dose is missed and a 30-day interval between doses is exceeded, administer Advantage Multi® for Dogs immediately and resume the monthly dosing schedule. When replacing another heartworm preventative product in a heartworm prevention program, the first treatment with Advantage Multi for Dogs should be given within one month of the last dose of the former medication. Moreover, Bowman D. et al. (Parasites & Vectors (2016) 9:12) confirm that the efficacy of prevention was 100% when dogs are infected only 28 days after the last monthly treatment. When dogs receive consecutive doses of Advantage Multi® as prescribed, heartworm infections will be prevented throughout the monthly dosing interval after administration of several monthly doses.

Selamectin spot-on is commercialized by Zoetis (Revolution®) to prevent of heartworm disease caused by Dirofilaria immitis, and to treat and control ear mite (Otodectes cynotis) infestations. The recommended minimum dose is 2.7 mg selamectin per pound (6 mg/kg) of body weight. Once again, this product is constraining because it has to be applied every month.

US2013231371 relates to a spot-on pesticidal composition comprising between about 0.25% to about 60% (w/w) pyrethroid and about 0.01% to about 10% (w/w) macrocyclic lactone. The composition is applied every four weeks.

WO2016161369 describes a composition of one depsipeptide and one macrocyclic lactone used as a treatment or prophylaxis of a parasitic infection wherein the parasitic infection comprises a parasite that is resistant to treatment or prophylaxis with the macrocyclic lactone alone, the parasite being Dirofilaria immitis. The administration of the cyclic depsipeptide and macrocyclic lactone is five times at monthly intervals. According to this patent application, the use of moxidectin alone cannot prevent from Dirofilaria immitis. Moreover, the application specifies that, in recent years an increased number of lack of efficacy cases have been reported, in which dogs develop mature heartworm infections despite receiving monthly prophylactic doses of macrocyclic lactones drugs and that an increasing number of cases of dogs that tested heartworm antigen positive while receiving heartworm preventive medication which speculates that Dirofilaria immitis has developed resistance to heartworm preventives.

Main weaknesses of the existing products and the latest developments are the lack of efficiency, the no long acting efficacy due to the administration mode (spot-on), the essential monthly applications of spot-on compositions, repeated during at least 4 months, to product a long acting composition which is endectocide and protects against heartworm. The long-acting products are injectable and not spot-on products.

Indeed, none of the prior art documents discloses a composition according to the present invention, comprising moxidectin or a salt thereof, which is useful to treat and/or prevent endoparasites (more particularly heartworms) and ear mites infestations in non-human mammals, which is long acting (at least 3 months) and easily topically applied, and one single application is sufficient and with optimized plasmatic concentration of drug to minimize long term toxicity issue in animal.

### SUMMARY OF THE INVENTION

Consequently, in order to overcome the foregoing problems including the recommendation of monthly applying the spot-on composition to treat heartworm or to inject a long-acting product, in order to produce a composition with an increased killing efficiency against endoparasites and ear mites, eradicate dirofilariasis, and provide more predictable performances, there is a need in the art for a new composition and a new method of treatment which affords improved absorption and bioavailability, at a lower maximum plasma concentration.

Therefore, the present invention aims to provide a novel use of a composition comprising moxidectin which protects against endoparasites like worms (roundworms, hookworms, heartworms, tapeworms and whipworms) and ear mites, which is very easy to administer (spot-on or line-on) and is able to maintain an effective plasma concentration over a long period, with a unique topical application.

The use of a composition comprising moxidectin according to the present invention has numerous advantages compared to prior art. It is safe, not toxic, chemically stable and well accepted. Only one active ingredient is necessary to treat and/or prevent against Dirofilaria immitis and ear mites, no synergetic association of two active ingredients (or more) is needed, unlike the existing products. The formulation is therefore easy to manufacture. There are neither relevant local (no red blotches, no hair loss, no itching, no scaling, limited cosmetic effect . . . ) nor general negative clinical signs (biochemistry/biology) due to the use of the composition, but an acceptable local and general tolerance by the non-human mammal.

This kind of “one shot formulation” has been optimized in order to achieve good stability and good spreadability without any solid deposits on furs, by carefully choosing the active ingredient, the excipients, the administration form and the concentrations.

Unlike the recommendations or prior art products, only one single topical application of the composition according to the present invention is useful for efficacy during several months. The composition has a good dermal permeation of moxidectin (higher than market products).

Moreover, the use of the composition according to the present invention allows to have a long half-life of the moxidectin in plasma/tissues fat, and to be well absorbed, well distributed. Therefore, it is very efficient against endoparasites, such as worms, well metabolized and well eliminated by the body (bioerodible, bioresorbable).

The composition used in the present invention is also ready-to-use. It is easy to use it as a veterinary medicine: the user neither needs to prepare any suspension or solution, nor need to measure/calculate and extract the convenient amount of drug from a syringe, according to the weight of the animal, hence, the composition can be easily topically applied by the animal owner, in a single application, and no more by a veterinarian. There are no more risks due to injection (infections). It is a ready-to-use spot-on/line-on composition. Consequently, there is no risk of dose error. The dosage regimen is perfectly controlled thanks to the ready-to-use pipette, resulting in a better observance of the treatment. The composition combines the long acting efficiency of the injectable existing products with the administration ease of a topical product.

Moreover, the use of the composition according to the present invention allows to obtain a good pharmacokinetic profile in mammals, especially, in dogs and cats and to have a tremendous effectiveness against gastrointestinal and respiratory nematodes.

Moreover, the topical (spot-on, line-on) administration increases the composition efficiency: there is a better protection than with a monthly oral administration (Blagburn, 2011). More particularly, the “line-on” application allows the control of the active ingredients diffusion: the composition is administered externally against the grain of the animal and applied continuously.

The inventors surprisingly found that, unlike the prior art conclusions, a so frequent monthly application is not useful to achieve an efficient plasma concentration: the use of the composition according to this present application is efficacious during 3 months at least. Moreover, there is no drug accumulation in the animal, therefore the toxicity and ecotoxicity (environment friendly) are reduced.

Therefore, the problem solved by the present invention, is to provide a new method of treatment comprising the administration of a veterinary or pharmaceutical composition which is easily topically applied and used to treat and/or prevent parasites infestations in a non-human mammal, more particularly heartworm and ear mites, which is long acting, which has a higher efficacy than the existing commercial products, and allows an efficient plasma concentration during at least three months, or more.

In a first aspect, the aim of the present invention is to provide a veterinary or pharmaceutical composition comprising moxidectin, or a salt thereof, for use in preventing and/or treating parasites infestations in a non-human mammal, characterized in that the composition is topically administered every 3 to 9 months. The veterinary or pharmaceutical composition as defined herein can be considered as a long acting composition.

A further object of the invention is a method of treatment and/or prevention of parasites infestations in a non-human mammal, comprising topically administering every 3 to 9 months to said non-human mammal a veterinary or pharmaceutical composition comprising moxidectin, or a salt thereof.

### BRIEF DESCRIPTION OF THE FIGURE

FIG. 1 is a graph showing the pharmacokinetic profile of a composition according to the present invention (formulation 1) compared to the pharmacokinetic profile of the injectable product Proheart 6® administered at the dose of 0.17 mg/kg (see example 7).

FIG. 2 is a graph showing the plasma concentration-time profiles observed after a single topical of the compositions according to the present invention (formulations 7-10) in Beagle dogs.

### DETAILED DESCRIPTION OF THE INVENTION

In a first aspect, this invention relates, to a veterinary or pharmaceutical composition comprising moxidectin, or a salt thereof, for use in preventing and/or treating parasites infestations in a non-human mammal, characterized in that the composition is topically administered every 3 to 9 months.

Within the context of invention “pharmaceutical composition” refers to a composition containing drugs used to treat and/or diagnose and/or cure and/or prevent diseases. Furthermore, a drug is any substance or combination of substances (composition) presented as having properties to treat and/or prevent disease(s) in human beings; or any substance or combination of substances which may be used in, or administered to human beings either with a view to restoring, correcting or modifying physiological functions by exerting a pharmacological, immunological or metabolic action, or to making a medical diagnosis (according to the Directive 2004/27/EC).

According to the FDA glossary, within the context of invention “pharmaceutical composition” also refers to a “drug product” which is the finished dosage form that contains a drug substance, generally, but not necessarily in association with other active or inactive ingredients. A drug is defined as a substance recognized by an official pharmacopoeia or formulary, a substance intended for use in the diagnosis, cure, mitigation, treatment, or prevention of disease, a substance (other than food) intended to affect the structure or any function of the body, a substance intended for use as a component of a medicine but not a device or a component, part or accessory of a device (biological products are included within this definition and are generally covered by the same laws and regulations, but differences exist regarding their manufacturing processes-chemical process versus biological process-).

According to the present invention the term “veterinary” has the same definition as “pharmaceutical”, but adapted to animals (meaning non-human beings): “animal” means any living stage of any member of the animal kingdom except human beings. More precisely, a “veterinary drug” (or medicine or composition) according to the present invention means any substance or mixture of substances which is used, or is manufactured, sold or represented as suitable for use, in the diagnosis, treatment, control, eradication, mitigation or prevention of disease or abnormal physical or mental state or the symptoms thereof in an animal; or restoring, correcting, controlling, or modifying any physical, mental or organic function in an animal.

As used herein, the term “comprising” may include the presence of other active(s) ingredient(s) and any other excipients. The term “comprising” may also designate “consisting of” and vice versa.

In another embodiment, the veterinary or pharmaceutical composition for use according to the invention comprises moxidectin or a salt thereof as a sole active ingredient. It means that the composition of the invention comprises only one active ingredient, which is moxidectin or a salt thereof, thereby not comprising a combination of active ingredients. Thus, an object of the invention is a veterinary or pharmaceutical composition comprising moxidectin or a salt thereof as a sole active ingredient, for use in preventing and/or treating a parasite infestation in a non-human mammal, characterized in that the composition is topically administered every 3 to 9 months. A further object is a veterinary or pharmaceutical composition comprising an active ingredient consisting of moxidectin or a salt thereof, for use in preventing and/or treating a parasite infestation in a non-human mammal, characterized in that the composition is topically administered every 3 to 9 months.

Moxidectin is a macrocyclic lactone and macrocyclic lactones are classified in two groups of structurally related molecules: milbemycins and avermectins (ivermectin, doramectin, abamectin, eprinomectin and selamectin). The first veterinary macrocyclic lactone, ivermectin, was introduced as an antiparasitic drug in 1981 and its tremendous efficacy against nematodes and arthropods took parasite control to a new level. Heartworms (L3 and L4 larvae) are particularly sensitive to macrocyclic lactones.

However, it was early discovered that certain breeds of dog with MDR-1 deficiency are highly susceptible to the toxic effects of macrocyclic lactones. A well-known sensitive breed are collie dogs. Therefore, the maximal tolerated dose in collie and related dogs dictates the acceptable therapeutic dose range.

More particularly, ivermectin has a narrow safety margin. Therefore, its use is restricted to the prevention of heartworm (6 μg/kg per os or 80 μg/kg topically). Milbemycin oxime is also used in the control of French heartworm (Angiostrongylus vasorum), when applied four times at weekly intervals. Moxidectin is applied orally for the prevention of heartworm (3 μg/kg) and topically for the control of and gastro-intestinal and respiratory nematodes. Topical application is safe in collie dogs.

Moxidectin has a tremendous efficacy against gastrointestinal and respiratory nematodes (adults, immature adults and L4 stages). The recommended monthly application is also effective against respiratory nematodes.

Moxidectin (or milbemycin B) has the structural formula (milbemycin B, cas n° 11350706-5, molecular weight 639.8 g·mol⁻¹):

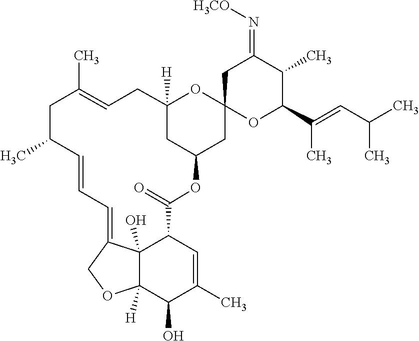 [SMILES: CO/N=C1\C[C@]2(C[C@@H]3C[C@@H](C/C=C(\C)C[C@@H](C)/C=C/C=C4\CO[C@@H]5[C@H](O)C(C)=C[C@@H](C(=O)O3)[C@]45O)O2)O[C@H](/C(C)=C/C(C)C)[C@H]1C]

More particularly, for the use according to the present invention, the amount moxidectin, or a salt thereof, is comprised between 1.0 and 4% weight/volume (% w/v) of the total composition volume, especially between 1.5-3.5% w/v, especially between 2.0-3.5% w/v, especially between 2.0-3.0% w/v, especially between 2.5-3.0% w/v. More preferably, moxidectin has a concentration comprised between 2.0-3.5% w/v, more preferably moxidectin is present in an amount of 2.5% w/v of the total composition, and even more preferably in an amount of 3.0% w/v.

Within the context of invention, % weight/volume or % w/v is the mass concentration defined as the mass (g) of a constituent divided by the volume (100 mL) of the total composition. For example, 25 mg/mL is equivalent to 2.5% w/v.

The term moxidectin also comprises its pharmaceutically acceptable salts. The salt can be hydrochloride, hydrobromide, phosphate, nitrate sulfate, fumarate, citrate, tartrate, acetate, maleate, toluenesulfonate, methanesulfonate, or mixtures thereof and the like.

Furthermore, the composition for use according to the present invention may further include any of the following other excipients in a pharmaceutically acceptable amount such as, for example, one or more: film forming, solvents, antioxidants, flowing agents, lubricants, diluents, preservatives, crystallization inhibitors, colloids, adhesives, thickeners, thixotropic agents, penetrating agents, stabilizers, solubilizing agents, fluidizing agents, complexing agents, vitamins, minerals, antiseptic agents, or combinations thereof. More generally, the active ingredients may be combined with any liquid additives corresponding to the usual technologies of formulation development. More preferably, the composition according to the present invention further comprises excipients such as solvents and antioxidants.

An excipient, or auxiliary substance, refers to any drug component which is not an active substance (such as adjuvants, stabilizers, diluents, antioxidants, antimicrobial preservatives . . . ), according to pharmacopeias.

Solvents (polar, apolar, protic, aprotic) may be selected from: benzyl alcohol, isopropyl alcohol, medium chain triglycerides (having a chain between 6 and 12 carbons atoms), propylene carbonate, dipropylene glycol monomethyl ether (DPGME, Dowanol®), dimethylsulfoxyde (DMSO), N-octyl-2-pyrrolidone (NOP), N-methyl-2-pyrrolidone (NMP), transcutol P or HP® (2-(2-ethoxyethoxy)ethanol or highly purified diethylene glycol monoethyl ether), acetone, 2-butanone, 3-methyl-2-butanone, cyclohexanone, acetonitrile, xylene, chlorobenzene, methylene chloride, chloroform trichloroethane, benzaldehyde ethylene chloride, sulfolane, methyl tert-butyl ether, dibutyl ether, ethyl acetate, acetate propyl methacrylate, amyl acetate, propyl acetate, dimethylformamide (DMF), dimethylacetamide (DMAC), propylene diethylcarbonate, ethylene carbonate, acetonitrile, triethylamine, pyridine, methanol, ethanol, isopropanol, hexafluoroisopropanol, carboxylic acids such as formic acid and acetic acid, primary and secondary amines, propylene alkyl ether, ethylene alkyl ether, polyglycol alkyl ether, di polyglycol allyl, polypropylene glycol, polyethylene glycol, and mixtures thereof. The preferred solvents are benzyl alcohol, isopropyl alcohol, medium chain triglycerides, propylene carbonate, dipropylene glycol monomethyl ether, and the mixtures thereof. The solvent or the mixture of solvents are present in an amount of between 95.5% w/v and 98.99% w/v of the total composition, more preferably is present in an amount between 96.95% w/v and 97.45% w/v of the total composition.

According to the present invention, the amount of propylene carbonate and DPGME is comprised between about 15% and 20% w/v of the composition. The preferred amount of propylene carbonate is 16% w/v. The preferred amount of DPGME is 20% w/v. The amount of benzyl alcohol and isopropyl alcohol is comprised between about 75.5% w/v and 83.99% w/v. The amount of medium chain triglycerides is between 10% and 40% w/v of the composition, preferably between 16% and 32% w/v, more preferably 16% or 32% w/v.

Antioxidants may be selected from: 2,6-di-tert-butyl-4-methylphenol (butyl hydroxytoluene or BHT), vitamin E (DL-alpha-tocopherol, E307), vitamin E phosphate, vitamin A, ascorbic acid (vitamin C), vitamin B12, polyphenols, butyl hydroxyanisol (BHA), propylgallate, tocopherol, ascorbic acid, citric acid, di-alpha-tocopheryl phosphate, beta-carotene, carotenes, carotenoids, flavonoids, sulfate compounds, L-cysteine, thiodipropionic acid, thiolactic acid, monothioglycerol, propyl galate sodium metabisulfite, sodium formaldehyde, sulfoxylate acetate, and mixtures thereof. The preferred antioxidant is BHT. Antioxidants/antioxidant are/is present in an amount of between 0.001 and 0.5%, more preferably is present in an amount of 0.05% w/v of the composition.

More preferably, the antioxidant is BHT and the solvents are benzyl alcohol, isopropyl alcohol, propylene carbonate, medium chain triglycerides and/or DPGME.

More preferably, for the use according to the present invention, film forming agents are selected from polyvinylpyrrolidones and derivatives thereof, polysaccharides, cellulose and derivatives of cellulose such as ethylcellulose, gums, polyvinyl alcohols, acrylic polymers and copolymers, polyacrylamides and mixtures thereof. Film forming agent is present in an amount comprised between 0 and 2% w/v, more preferably is present in an amount of 1% w/v of the composition.

Preservatives may be selected from: methyl p-hydroxybenzoate, ethyl p-hydroxybenzoate, phenol, sorbic acid, cresol and chlorocresol, and mixtures thereof.

Illustrative thickening agents include methylcellulose, ethylcellulose, hydroxyethyl cellulose, sodium carboxymethyl cellulose, hydroxypropyl cellulose, polyvinylpyrrolidone, and mixtures thereof.

Illustrative complexing agents include EDTA and salts thereof, phosphate, nitrate, acetate, citrate, and mixtures thereof.

Illustrative antiseptics include methyl p-oxybenzoate, propyl p-oxybenzoate, PHB ester, chlorobutanol, benzyl alcohol, butanol, butane-1,3-diol, chlorohexidin salts, benzoic acid and its salts, sorbic acid, and mixtures thereof.

The excipients as disclosed herein may be classified in one or several categories. For instance, the films forming agents, such as ethylcellulose, may be also considered as thickening agents.

A preferred film forming agent and/or thickening agent is ethylcellusose, preferably ethylcellulose of grade 7, 20, or 100. An ethylcellulose of grade 7 has a viscosity comprised between 6 and 8 mPa·s. An ethylcellulose of grade 20 has a viscosity comprised between 18 and 22 mPa·s. An ethylcellulose of grade 100 has a viscosity comprised between 90 and 100 mPa·s. In a preferred embodiment, the amount of ethylcellulose is between 0.1% and 2% w/v of the composition, preferably between 0.5% and 1.5% w/v, more preferably 1.0% w/v.

A preferred veterinary or pharmaceutical composition for use according to the invention comprises:
about 3 w/v % moxidectin,
about 1 w/v % ethylcellulose,
about 0.05 w/v % BHT,
about 32 w/v % medium chain TG, and
benzyl alcohol QS 1 mL.

In a very particular embodiment, the composition for use may further comprise one or more additional active substances like ectoparasitic or endoparasitic control agents, antibiotics, non-steroidal anti-inflammatory drugs.

An active substance refers to any substance intended to be used to prepare a medicine, and, when it is used in the manufacture of the medicine, becomes an active substance of this medicine, such substances are intended to supply a pharmacological activity or another direct effect for the disease diagnosis, healing, attenuation, treatment or prevention or to produce an effect on the body structure and function (as defined in pharmacopeias).

As an example of ectoparasitic agents, it may be cited organochlorines, organophosphates, formamidines, amidines, carbamates, pyrethroids (cypermethrin, deltamethrin, lumethrin, permethrin, cyfluthrin, flumethrin, metofluthrin, momfluorothrin), pyrethrins, phenylpyrazoles (fipronil, pyriprole), benzoylureas, neonicotinoids (dinotefuran, imidacloprid, nitenpyram), oxadiazines, spinosyns (spinosad, spinetoram), isoxazolines (afoxolaner, fluralaner, lotilaner, sarolaner), cholinesterase inhibitors, insect growth regulators (fluazuron, methoprene, pyriproxifen, triflumuron, lufenuron, novaluron, chlorfluazuron, hydroprene), and the like, or mixtures thereof.

As an example of endoparasitic agents, it may be cited: benzimidazoles (enbendazole, oxfendazole, albendazole, triclabendazole), imidazothiazoles (levamisole, tetramisole), pyrimidines (pyrantel, pyrantel tartrate), isoquinolines (praziquantel, epsiprantel), salicylanilides (closantel, niclosamide, oxyclozanide, rafoxanide), tetrahydropyrimidines, amino-acetonitrile derivatives, depsipeptides, spiroindoles, macrocyclic lactones (ivermectin, selamectin . . . ) and the like, or mixtures thereof. The preferred endoparasitic agent is ivermectin, or selamectin.

According to the present invention, non-human mammals refers to companion animals, or pets, or any domesticated animals, and includes, without any limitation, dogs, puppies, cats, kitten, rabbits, sheep, goats, pigs, cows, gerbils, horses, mice, ferrets, hamsters, horses, and the like. In a preferred embodiment, the non-human mammal is a pet, such as a canine, or such as a cat, more preferably is a dog. The dog can be a small size dog, a medium size dog or a large size dog.

“Preventing and/or treating” as used herein include the control, the reduction, the progression slowing, the eradication, the cure and/or avoid parasites infestations.

In a particular embodiment, the composition is administered, more precisely topically administered, every 3 to 6 months, more preferably 3 to 9 months, more preferably every 4 to 9 months, more preferably every 5 to 9 months, more preferably every 6 to 9 months, more preferably every 6 to 8 months, more preferably, every 3 months, more preferably every 4 months, more preferably every 5 months, more preferably every 6 months, more preferably every 7 months, more preferably every 8 months and more preferably every 9 months. It has as an efficacy of 3, 6 months or more, up to 9 months. “Efficacy” used herein refers to a therapeutically effective amount of the active substance to treat and/or prevent diseases. Examples of effective dosages (pipette volume), in dogs, are: For 30 mg/mL of moxidectin, in a pipette (pipette volume):
X small dogs (equal of <4 kg): 0.4 mL,
small dogs (>4-10 kg): 1 mL,
medium dogs (>10-25 kg): 2.5 mL,
large dogs (>25-40 kg): 4 mL,
X large dogs (>40-60 kg): 6 mL,
XX large dogs>75 kg (>165 lbs: 7.5 mL.

For 20 mg/mL of moxidectin, in a pipette (pipette volume):
X small dogs (equal of <4 kg): 0.5 mL,
small dogs (>4-10 kg): 1.25 mL,
medium dogs (>10-25 kg): 3.125 mL,
large dogs (>25-40 kg): 5 mL,
X large dogs (>40-60 kg): 7.5 mL,
XX large dogs>75 kg (>165 lbs): 11.25 mL.

According to the invention, the composition is intended to be topically administered every 3 to 9 months. According to a particular embodiment, the composition is administered in a single dose to the non-human mammal at a time T0 and is then again administered at a time T1, at least 3, 4, 5, 6, 7, 8 or 9 months later. Particularly, the treatment can be repeated every 3 months, every 4 months, every 5 months, every 6 months, every 7 months, every 8 months, or every 9 months. According to this embodiment, the term “every” means a repeated treatment.

In another particular embodiment, the composition is administered in a single dose to the non-human mammal at a time T0 and is not repeated later. According to this particular embodiment, the term “every” means a unique treatment in which the composition is effective up to 3, 4, 5, 6, 7, 8, and preferably up to 9 months. Thus, the present invention also relates to a veterinary or pharmaceutical composition as disclosed herein comprising moxidectin or the salt thereof, preferably as a sole active ingredient, for use in preventing and/or treating a parasite infestation in a non-human mammal. Preferably, the veterinary or pharmaceutical composition is a 3, 4, 5, 6, 7, 8, or 9 months long acting composition.

In a preferred embodiment, the plasma concentration in the animal dosed at 2.5 mg/kg of moxidectin is above 0.025 ng/mL, for a period of at least 3 months, or of 3 months, for a period of at least 6 months, or of 6 months, or for a period of at least 7 months, or of 7 months, or for a period of at least 8 months, or of 8 months, or for a period of at least 9 months, or of 9 months.

In a particular embodiment of the present application, the moxidectin is administered in an amount comprised between 1.5 mg/kg of body weight (BW) and 15 mg/kg of BW, particularly between 1.5 mg/kg of body weight (BW) and 3.5 mg/kg of BW, more particularly 2.5 mg/kg of BW, even more preferably, 3.0 mg/kg of BW.

The composition for use according to the present invention is in the form of a liquid solution, semi-liquid solution, suspension, paste, cream, foam, ointment, or gel. The composition is administered topically, by spot-on route, more especially line-on administration. The line-on application is preferred to limit the risk of product loss and to enhance skin absorption of moxidectin.

“Administered” herein, and more precisely “line-on”, means the composition is applied on the skin of the animal, from the base of the tail along the spine to the shoulder blades, or from the middle of the back along the spine to the shoulder blades, or less: the length of the “line-on” application can for example be 30 cm, or 20 cm, or 15 cm, or 10 cm, or 5 cm, the preferred length being 10 cm. Composition is formulated as a unit dose adapted to the weight and/or size of the animal, and the entire dose is applied to the animal. Thanks to the line-on application method, the amount of diffused moxidectin through the animal skin is known and controlled.

According to the present invention, parasites infestations are caused by worms, especially, by nematodes (endoparasites), and/or mites (ectoparasites), more especially by Dirofilaria immitis, and/or ear mites.

In an embodiment according to the present invention, the ectoparasite is a mite. Cat mites are from the families Demodicidae, Psoroptidae, Sarcoptidae, Cheyletidae, Dermanyssidae and/or Trombiculidae:
Demodex spp.: cati
Otodectes spp.: cynotis
Notoedres spp.: cati
Sarcoptes spp.: scabiei
Cheyletiella spp.: blakei, parasitovorax
Dermanyssus spp.: gallinae
Neotrombicula spp.: autumnalis

The preferred mite is Otodectes spp (ear mite).

Cat and dog mites are from the families Boopidae, Cheyletidae, Psoroptidae, Sarcoptidae, Demodicidae, Dermanyssidae and/or Trombiculidae:
Cheyletiella spp.: yasguri
Otodectes spp.: cyanoti
Sarcoptes spp.: scabiei
Notoedres spp.: cati
Demodex spp.: canis
Dermanyssus spp.: gallinae
Neotrombicula spp.: autumnalis.

The preferred mite is Otodectes spp (ear mite).

The composition according to the invention can also be used to treat, apart from heartworms, against endoparasites, more especially hookworms, flatworms, tapeworms, whipworms, more especially gastro-intestinal nematodes, cardio-pulmonary nematodes.

In one embodiment of the invention, the endoparasite is a nematode and/or a trematode in circulatory system. Cat worms are from the families Schistosomatidae and/or Filarioidae:
Schistosoma spp.: japonicum, rodhaini
Dirofilaria spp.: immitis
Brugia spp.: pahangi, malayi

The preferred circulatory system worm is Dirofilaria spp. (immitis: heartworm).

Dog worms are from the families Schistosomatidae, Metastrongylidea and/or Filarioidae:
Angiostrongylus spp.: vasorum
Schistosoma spp.: japonicum, spindale, incognitum,
Heterobilharzia spp.: americana
Dirofilaria spp.: immitis
Brugia spp.: pahangi, malayi.

The preferred circulatory system worm is Dirofilaria spp. (immitis: heartworm).

In another embodiment of the invention, the endoparasite is a nematode, cestode, trematode and/or acanthocephalans in small intestine. Cat worms are from the families Ascaridoidea, Ancylostomoidea, Rhabditoidea, Diphyllobotrhiidae, Dilepididae, Taeniidae, Diphyllobothriidae, Mesocestoididae, Diplistomatidae, Heterophydiae, Echinostomatidae, Pliganthorynchidae and/or Trichuroidea:
Toxoscaris spp.: leonina
Toxocara spp.: mystax, malayensis
Ancylostoma spp.: braziliense, ceylanicum, tibaeforme
Uncinaria spp.: stenocephala
Strongyloides spp.: stercocoralis, planiceps, felis, tumefaciens
Diphyllobothrium spp.: latum, caninum
Echinococcus spp.: multilocularis, oligarthrus
Spirometra spp.: masoni, mansonoides, erinacei
Taenia spp.: taeniaeformis
Mesocestoides spp.: lineatus
Alaria spp.: alata, minessotae, marcianae
Heterophyes spp.: heterophyes, nocens
Metagonimus spp.: yokogawai
Apophallus spp.: donicum, mulhingi
Cryptocotyle spp.: lingua
Echinochasmus spp.: perfoliatus
Euparyphium spp.: melis
Nanophyetus spp.: salmincola
Macracanthorhynchus spp.: hirudinaceus, catalinum
Onicola spp.: campanulatus
Trichinella spp.: serrata, vulpis, campanula.

Dog worms are from the families Ascaridoidea, Ancylostomoidea, Rhabditoidea, Diphyllobotrhiidae, Dilepididae, Taeniidae, Diphyllobothriidae, Mesocestoididae, Diplistomatidae, Heterophydiae, Echinostomatidae, Pliganthorynchidae and/or Trichuroidea:
Toxocara spp.: canis, leonina
Ancylostoma spp.: caninum, braziliense, ceylanicum
Uncinaria spp.: stenocephala
Strongyloides spp.: stercoralis
Diphyllobothrium spp.: latum, caninum
Echinococcus spp.: granulosus, quinus, orteleppi, multilocularis, vogeli
Spirometra spp.: masoni, mansonoides
Taenia spp.: hydatigena, multiceps, ovis, pisiformis, serialis, crassiceps
Mesocestoides spp.: lineatus
Alaria spp.: alata, americana, canis, michiganensis
Heterophyes spp.: heterophyes, nocens
Metagonimus spp.: yokogawai
Apophallus spp.: donicum, mulhingi
Cryptocotyle spp.: lingua
Echinochasmus spp.: perfoliatus, ilocanum
Nanophyetus spp.: salmincola.

In another embodiment of the invention, the cat and dog endoparasite is a nematode in subcutaneous tissues: Dirofilariae spp.: repens.

In this application, each genera include all the associated species.

In a preferred embodiment, the parasite infestation is dirofilariasis.

A more preferred embodiment of the invention relates to a veterinary or pharmaceutical composition comprising moxidectin or a salt thereof, preferably as a sole active ingredient, for use in preventing and/or treating a parasite infestation, preferably dirofilariasis in a non-human mammal such as a dog or a cat, characterized in that the composition is topically administered in said non-human mammal every 3 to 9 months, preferably every 3 to 6 months, more preferably every 3 months in a single take. Preferably, the amount of moxidectin or a salt thereof is comprised between 1.5 mg/kg and 3.5 mg/kg BW, more preferably about 3 mg/kg BW.

Another object of the present invention is a method of treatment and/or prevention of parasites infestations in a non-human mammal, comprising topically administering every 3 to 9 months to said non-human mammal, a veterinary or pharmaceutical composition comprising moxidectin or a salt thereof.

A more preferred embodiment of the invention is a method for preventing and/or treating dirofilariasis in a dog or a cat, comprising topically administering in a single take a composition comprising moxidectin or a salt thereof, preferably as a sole active ingredient, in an amount comprised between 1.5 mg/kg and 3.5 mg/kg BW, more preferably about 3 mg/kg BW, to said dog or cat every 3 to 9 months, preferably every 3 to 6 months, more preferably every 3 months.

Another object of the present invention is a use of moxidectin or a salt thereof for the manufacture of a veterinary or pharmaceutical composition for preventing and/or treating a parasite infestation in a non-human mammal, in which composition is topically administered to said non-human mammal every 3 to 9 months.

A more preferred embodiment of the invention is a use of moxidectin or a salt thereof, preferably as a sole active ingredient, for the manufacture of a veterinary or pharmaceutical composition for preventing and/or treating dirofilariasis in a dog or a cat, in which said composition comprises an amount of moxidectin comprised between 1.5 mg/kg and 3.5 mg/kg BW, more preferably about 3 mg/kg BW, and said composition is topically administered to said dog or cat every 3 to 9 months, preferably every 3 to 6 months, more preferably every 3 months, in a single take.

Another object of the present invention is a kit useful in preventing and/or treating parasites in a non-human mammal comprising a composition as described above, within a pipette, equipped with an applicator tip. The pipette can have five dosages: 0.4 mL, 1 mL, 2.5 mL, 4 mL and 6 mL.

All embodiments described above for the use of the composition also apply to the method of treatment and to the kit comprising said composition, as described below.

Described herein below are examples: preparation of compositions according to the present invention and their use. These examples are illustrative and in no way limiting.

### EXAMPLES

### Example 1: Formulation 1

In a manufacturing container the moxidectin solution according to the composition depicted below has been prepared. Under stirring a part of benzyl alcohol, the butyl hydroxyl toluene (BHT), the propylene carbonate and the moxidectin have been added. Then, the volume has been completed with benzyl alcohol.

| | Amount (% w/v = 10 x mg/mL) |
|---|---|
| Ingredients | Batch A38 |
| Moxidectin | 3 |
| BHT | 0.05 |
| Propylene carbonate | 16 |
| Benzyl alcohol | QS 100 (or QS 1 mL) |

### Example 2: Formulation 2

In a manufacturing container the moxidectin solution according to the composition depicted below has been prepared. Under stirring a part of isopropyl alcohol, BHT, the dipropylene glycol monomethyl ether (DPGME) and the moxidectin have been added. Then, the volume has been completed with isopropyl alcohol.

| | Amount (%) |
|---|---|
| Ingredients | Batch A40 |
| Moxidectin | 3 |
| BHT | 0.05 |
| Dowanol ® DPGME | 20 |
| Isopropyl alcohol | QS |

### Example 3: Formulation 3

The same process as in example 1 has been used.

| | Amount (%) |
|---|---|
| Ingredients | Batch A14 |
| Moxidectin | 2.5 |
| BHT | 0.05 |
| Propylene carbonate | 16 |
| Benzyl alcohol | QS |

### Example 4: Formulation 4

The same process as in example 2 has been used.

| Ingredients | Amount (%) |
|---|---|
| Moxidectin | 2.5 |
| BHT | 0.05 |
| Dowanol ® DPGME | 15 |
| Isopropyl alcohol | QS |

### Example 5: Formulation 5

In a manufacturing container the moxidectin solution according to the composition depicted below has been prepared. Under stirring a part of benzyl alcohol, the butyl hydroxyl toluene, the propylene carbonate, the polyvinylpyrrolidone and the moxidectin have been added. Then, the volume has been completed with benzyl alcohol.

| | Amount (mg/mL) | |
|---|---|---|
| Ingredients | Batch Z14 | |
| Moxidectin | 30 | mg/mL |
| BHT | 0.5 | mg/mL |
| Polyvinylpyrrolidone (PVP K30) | 10 | mg/mL |
| Propylene Carbonate | 160 | mg/mL |

| | |
|---|---|
| Benzyl Alcohol | QS 1 mL |

### Example 6: Formulation 6

In a manufacturing container the moxidectin solution according to the composition depicted below has been prepared. Under stirring a part of isopropyl alcohol, the butyl hydroxyl toluene, the DPGME, the polyvinylpyrrolidone and the moxidectin have been added. Then, the volume has been completed with isopropyl alcohol.

| | Amount (mg/mL) | |
|---|---|---|
| Ingredients | Batch Z16 | |
| Moxidectin | 30 | mg/mL |
| BHT | 0.5 | mg/mL |
| Polyvinylpyrrolidone (PVP K30) | 10 | mg/mL |
| DPGME | 200 | mg/mL |

| | |
|---|---|
| Isopropyl alcohol | QS 1 mL |

### Example 7: Formulation 7

In a manufacturing container the moxidectin solution according to the composition depicted below has been prepared. Under stirring a part of benzyl alcohol, the butyl hydroxyl toluene (BHT), the medium chain triglyceride and the ethyl cellulose have been added. When the solution is homogeneous the moxidectin is added. Then, the volume has been completed with benzyl alcohol.

| | Amount (mg/mL) | |
|---|---|---|
| Lot | Batch A106 | |
| Moxidectin | 30 | mg/mL |
| Ethyl cellulose | 10 | mg/mL |
| BHT | 0.5 | mg/mL |
| Medium chain TG | 320 | mg/mL |

| | |
|---|---|
| Benzyl Alcohol | QS 1 mL |

### Example 8: Formulation 8

In a manufacturing container the moxidectin solution according to the composition depicted below has been prepared. Under stirring a part of benzyl alcohol, the butyl hydroxyl toluene (BHT), the medium chain triglyceride, PEG-35 Castoir oil and the moxidectin have been added. Then, the volume has been completed with benzyl alcohol.

| | Amount (mg/mL) | |
|---|---|---|
| Lot | Batch A73 | |
| Moxidectin | 30 | mg/mL |
| PEG-35 Castor Oil | 50 | mg/mL |
| BHT | 0.5 | mg/mL |
| Medium chain TG | 320 | mg/mL |

| | |
|---|---|
| Benzyl Alcohol | QS 1 mL |

### Example 9: Formulation 9

In a manufacturing container the moxidectin solution according to the composition depicted below has been prepared. Under stirring a part of benzyl alcohol, the butyl hydroxyl toluene (BHT), the medium chain triglyceride and the ethyl cellulose have been added. When the solution is homogeneous the moxidectin has been added. Then, the volume has been completed with benzyl alcohol.

| | Amount (mg/mL) | |
|---|---|---|
| Lot | Batch A107 | |
| Moxidectin | 30 | mg/mL |
| Ethyl cellulose | 10 | mg/mL |
| BHT | 0.5 | mg/mL |
| Medium chain TG | 160 | mg/mL |

| | |
|---|---|
| Benzyl Alcohol | QS 1 mL |

### Example 10: Formulation 10

In a manufacturing container the moxidectin solution according to the composition depicted below has been prepared. Under stirring a part of benzyl alcohol, the butyl hydroxyl toluene (BHT), the medium chain triglyceride and the ethyl cellulose have been added. When the solution is homogeneous the moxidectin and the PEG-35 castor oil have been added. Then, the volume has been completed with benzyl alcohol.

| | Amount (mg/mL) | |
|---|---|---|
| Lot | Batch A108 | |
| Moxidectin | 30 | mg/mL |
| Ethyl cellulose | 10 | mg/mL |
| PEG-35 Castor Oil | 50 | mg/mL |
| BHT | 0.5 | mg/mL |
| Medium chain TG | 160 | mg/mL |

| | |
|---|---|
| Benzyl Alcohol | QS 1 mL |

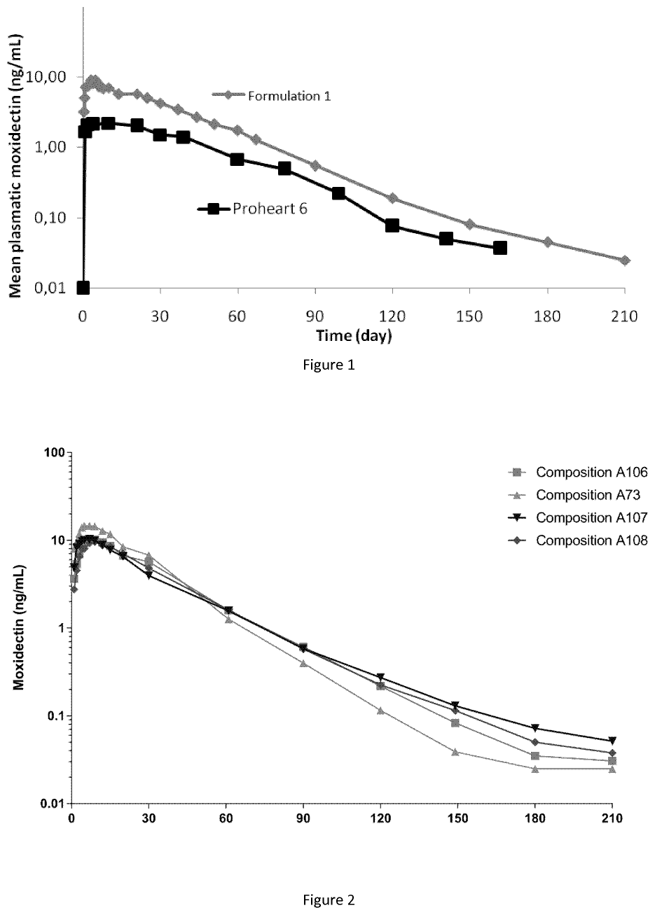

### Example 11: Pharmacokinetics in Dogs (See FIG. 1)

Samples of formulation 1 has been administered to three beagle dogs with a moxidectin dose of 3 mg/kg. Pharmacokinetics profile has been compared to the injectable product Proheart 6® administered at the dose of 0.17 mg/kg. The mean plasmatic profiles exhibit a maximum concentration (Cmax) above 5 ng/ml suitable for intestinal worms eradication and a plasmatic persistence similar or above Proheart®6 that allows heartworm prevention during 6 months. After a unique topical application, most endoparasites sensitive to moxidectin can be targeted.

### Example 12: Pharmacokinetics in Dogs (See FIG. 2)

The topical formulations A106, A73, A107, and A108 described in examples 7-10 have been tested in dogs. Groups of 8 beagle dogs have been treated with a single dose (0.1 ml/kg) of each formulation and blood sampled for pharmacokinetics. The topical formulations were well tolerated. The absorption was fast, sustained and significant. Sustained blood levels similar to Proheart (Example 1 and FIG. 1) were observed during more than 210 days post treatment.

1. A method for preventing and/or treating a parasite infestation in a non-human mammal, comprising topically administering every 3 to 9 months to said non-human mammal a veterinary or pharmaceutical composition comprising moxidectin, or a salt thereof.

2. A method for preventing and/or treating a parasite infestation in a non-human mammal, comprising topically administering every 3 to 9 months to said non-human mammal a veterinary or pharmaceutical composition comprising moxidectin, or a salt thereof as a sole active ingredient.

3. The method according to claim 1, wherein moxidectin has a concentration between 1-4 w/v %.

4. The method according to claim 1, wherein the non-human mammal is a pet.

5. The method according to claim 1, wherein the parasite infestation is caused by nematodes.

6. The method according to claim 5, wherein the nematode is Dirofilaria immitis.

7. The method according to claim 1, wherein the parasites infestation is caused by ear mites.

8. The method according to claim 1, wherein the composition is administered every 3 to 6 months.

9. The method according to claim 1, wherein the composition is administered every 9 months.

10. The method according to claim 1, wherein the topical administration of the composition is a line-on administration.

11. The method according to claim 1, wherein the topical administration of the composition is a spot-on administration.

12. The method according to claim 1, wherein the composition further comprises butyl hydroxytoluene as antioxidant.

13. The method according to claim 1, wherein the composition further comprises a solvent selected from the group consisting of benzyl alcohol, isopropyl alcohol, medium chain triglycerides, propylene carbonate, dipropylene glycol monomethyl ether, and mixtures thereof.

14. The method according to claim 1, wherein the composition comprises:
about 3 w/v % moxidectin,
about 1 w/v % ethylcellulose,
about 0.05 w/v % BHT,
about 32 w/v % medium chain TG, and
benzyl alcohol QS 1 mL.

15. The method according to claim 1, wherein the moxidectin is administered in an amount between about 1.5 mg/kg of body weight and 3.5 mg/kg of body weight.

16. The method according to claim 4, wherein the pet is a canine.

17. The method according to claim 15, wherein the moxidectin is administered in an amount of about 3.0 mg/kg of body weight.
